(19) United States
(12) Patent Application Publication
(10) Pub. No.: US 2026/0206702 A1
(43) Pub. Date: Jul. 23, 2026

(54) METHOD FOR ISOLATING PLANT STEM CELLS FROM PLANT LEAVES AND THE ASSOCIATED CELL LINES OBTAINED UTILISING THE METHOD

(71) Applicant: GREEN BIOACTIVES LIMITED, Penicuik (GB)

(72) Inventors: Yuan LI, Penicuik (GB); Samuel CASASOLA-ZAMORA, Penicuik (GB); Gary J. LOAKE, Penicuik (GB); David MCELROY, Penicuik (GB)

(21) Appl. No.: 18/881,428

(22) PCT Filed: Jul. 6, 2023
(86) PCT No.: PCT/EP2023/068729

§ 371 Date: Jan. 6, 2025

(30) Foreign Application Priority Data
Jul. 6, 2022 (GB) 2209919.6

(51) Int. Cl. A01H 4/00

(52) U.S. Cl. CPC A01H 4/005; A01H 4/008

(57) ABSTRACT

Disclosed herein is a method of isolating a stem cell line derived from a tissue from a primary meristem in a leaf of a plant, including the steps of sterilizing at least a part of the leaf containing the central or other vein; cutting through the central or other vein of the leaf to expose primary meristematic cells from the central or other vein inner vascular tissue; and culturing cells from said exposed cells, which circumvents the difficulties of surface sterilization when other types of explants are used. Also disclosed herein are stem cell lines obtained using the method.

This application is a National Phase entry of International Application No. PCT/EP2023/068729 under § 371 and claims the benefit of GB Patent Application No. 2209919.6, filed Jul. 6, 2022, which is hereby incorporated by reference in its entirety.

### FIELD OF THE DISCLOSURE

The present disclosure relates to methods for isolating stem cell(s) from a leaf of a plant, and establishing a stem cell line, and uses of the isolated stem cell(s) and stem cell line thereof.

### BACKGROUND

Plants are nature chemists, they are capable of producing a vast array of secondary metabolites (natural products) that form the cornerstone of many pharmaceutical, cosmetic, food and agricultural products. The source plant species used to produce natural product-containing extracts or for the isolation of specific purified natural products (NPs) are often slow growing, their populations limiting, with the concentration of the target molecule highly variable from plant to plant and its in vivo concentration routinely extremely low. This situation results in unmet demand for NPs and extracts, increasing the use of intensive farmland to grow NP-producing plants for industrial applications. Therefore, an alternative production system for plant NPs was required.

A potential solution is the utilization of in vitro cultured plant cells, isolated from the target NP producing plant species. The plant cell culture process consists in inducing callus formation from plant tissue or organs (explants) grown on a synthetic solid media in petri plates. The callus consists of a mass of dedifferentiated plant cells (DDCs). Subsequently, fragments of callus are inoculated in liquid media in flasks to generate a DDC suspension culture.

Plant cell culture provides an attractive alternative biological NP manufacturing route to whole plants. It is sustainable, efficient, independent of environmental conditions, standardized, provides a robust supply of product, is free of zoonotic viruses, can be optimized to produce commercially relevant levels of target metabolites, and is compatible with the pharmaceutical good manufacturing practice (GMP) regulatory guidelines.

However, this classical approach of culturing and using DDCs for NP production results in genetic and epigenetic damage to the plant cells. Consequently, DDCs exhibit major limitations including: slow growth rate, significant cell aggregation, increased stress sensitivity, unstable viability and limited NP production, limiting the widespread commercial use of this approach to all but the highest value applications.

To circumvent problems associated with the culturing of typical DDCs, Lee et al. (Cultured cambial meristematic cells as a source of plant NPs. Nature Biotechnology (2010) 28 (11), 1213-1217) isolated cultured cambium meristematic cells (CMCs). The cell suspension culture was derived from cambial tissue derived from secondary meristems within plant stems and twigs and showed potential as a platform to produce plant NPs. These cambium derived cells showed superior growth over long term culture, reduced cell aggregation, increased stress resistance and increased NP yields. Significantly, the cambium is a secondary meristem that is formed in stems and roots after the tissues of the primary plant body have differentiated. The cambium is responsible for increasing the diameter of stems and roots and for forming woody tissue (Fischer et al., 2019 Annu. Rev. Plant Biol. 70:293-319).

WO2007052876A1 discloses a method for isolating a cell line from a plant, wherein the isolated plant cell line is derived from the cambium obtained from the twig or stem of the plant. The method comprises collecting a tissue containing the cambium of the plant, culturing said tissue, thereby inducing a layer proliferated from the cambium secondary meristem without going through dedifferentiation into callus.

However, surface sterilization of explant from plant stem, twigs, root or storage root is difficult to perform successfully, especially for plant samples taken from the native environment, because these ecosystems have a rich microbial diversity. For example, due to their irregular surface architecture, explant samples taken from these plant organs are associated with high levels of endophytes, endosymbionts, often a bacterium or fungus, that resides within a plant for at least part of its life cycle without causing apparent disease (Hardoim et al. 2015. The Hidden World within Plants: Ecological and Evolutionary Considerations for Defining Functioning of Microbial Endophytes. Microbiol & Mol Biol Revs. 79 (3): 293-320). Therefore, the sterilization process itself needs to be systematically adapted to generate sterile cell cultures from different plant species, being both laborious and time consuming.

Other explants that may be employed in methods of isolating cells are, for example, the leaves of the plant. EP3942017A1 relates to the isolation of cell lines from a plant of the genus Melissa, by induction of callus formation from plant tissue including pieces of leaves or whole leaves on solid plant growth media and subsequent inoculation of a liquid plant growth media, to generate an associated plant cell suspension culture. However, the obtained Melissa DDCs exhibited major limitations including: slow growth rate, significant cell aggregation, increased stress sensitivity, unstable viability and low yields of NP production.

### SUMMARY

It is an aim of embodiments of the present invention to provide a method for isolating and generating a plant cell line that has not undergone dedifferentiation into callus, wherein the method is facile,
rapid, easier to standardize and circumvents the difficulties of surface sterilization when using plant secondary meristem tissue derived from the stem, twig or storage root as explant.

In one aspect, at least one embodiment of the invention relates to a method of isolating a stem cell line derived from a tissue from a primary meristem in a leaf of a plant, comprising the steps of:
(a) sterilizing at least a part of the leaf containing the central or other vein
(b) cutting through the central or other vein of the leaf to expose primary meristematic cells from the central or other vein inner vascular tissue; and
(c) culturing cells from said exposed cells in a cell induction medium.

That is, in one aspect of at least one embodiment of the invention, there is provided a method of isolating or obtaining at least one plant stem cell, the method comprising the steps of:
(a) sterilizing at least a part of a leaf containing a central or other vein;
(b) dissecting the central or other vein of the leaf to expose primary meristematic cells from the central or other vein inner vascular tissue; and
(c) obtaining at least one stem cell from the central or other vein inner vascular tissue.

In one embodiment, the stem cells are protophloem meristematic cells.

In one embodiment, the leaf is obtained from a plant grown or growing in a vertical farm.

The method of isolating a stem cell line derived from a tissue form a primary meristem in a leaf of a plant according to at least one embodiment of the present invention allows the culturing and isolation of cells that do not dedifferentiate into or have not de-differentiated from a callus.

In the method according to at least one embodiment of the present invention, cells are cultured from a leaf explant. Preferably, the leaves (e.g. young leaves) are collected from plant species that naturally produce a high-value natural product.

Accordingly, the method may comprise further comprises culturing the at least one stem cell to obtain a stem cell line.

In one embodiment, the cell culture does not or does not substantially comprise dedifferentiated cells and/or protoplasts. In one embodiment, the method does not comprise the formation of a callus obtained from or substantially comprising dedifferentiated cells- and subsequently obtaining stem cells from such a callus.

The culturing step may further comprise:
(a) culturing the primary meristematic cells from the central or other vein inner vascular tissue in suspension; and
(b) optionally selecting high-performance cell lines, preferably in response to plant immune elicitors.

The initiation of the suspension culture may comprise incubating a plate comprising the isolated primary meristematic cells in a 24 h/0 h light/dark to 0 h/24 h light/dark cycle at between 16-30° C.

In another aspect of at least one embodiment of the invention, there is provided a plant stem cell(s) or plant stem cell line obtained or obtainable by the method of at least one embodiment of the invention.

In another aspect of at least one embodiment of the invention, there is provided a plant stem cell product or extract obtained or obtainable from the plant stem cell(s) of at least one embodiment of the invention.

In another aspect of at least one embodiment of the invention there is provided a plant or part thereof obtained or obtainable from the plant stem cell of at least one embodiment of the invention. Also included within the scope of at least one embodiment of the invention are plant products and/or extracts obtained or obtainable from the plant.

The stem cell line derived from a primary meristem in a leaf of a plant preferably has the following characteristics:
(a) it is formed of undifferentiated cells, preferably substantially genetically homogenous cells; and
(b) the cells of the line are morphologically characterized by the presence of multiple amyloplasts; and
(c) it is a fast-growing cell line; and/or
(d) optionally, having high phytochemical accumulation in response to elicitation.

In another aspect of at least one embodiment of the invention, there is provided a method for producing a plant natural product, the method comprising isolating or obtaining at least one plant stem cell or cells as described herein and isolating/collecting the product.

In another aspect of the at least one embodiment of invention, there is provided a method for producing a stem cell extract the method comprising isolating or obtaining at least one plant stem cell or cells as described herein and isolating/obtaining an extract.

In one embodiment, the method may comprise
(a) isolating at least one stem cell derived from a tissue from a primary meristem in a leaf of a plant; and
(b) establishing a stem cell line comprising the at least one isolated stem cell, preferably wherein the stem cell line is substantially genetically homogenous;
(c) optionally selecting a high-performance cell line; and/or
(d) optionally, eliciting the production or secretion of a plant natural product or extract; and
(e) collecting the plant natural product or extract.

The high-performance cell line may be characterized by a high-performance indicator or trait, selected from at least one of: cell size, proliferation, yield of a product and/or developmental potency, wherein preferably said improvement is relative to another/other cell/cells in the cell line.

Eliciting the production or secretion may comprise treating the suspension culture with a plant elicitor.

Plant natural compounds show a broad spectrum of activities against insects, fungi, bacteria and viruses. Accordingly, in one embodiment, the plant product may be an insecticide, a fungicide, an anti-bacterial agent or an anti-viral agent. Alternatively, the product may be a therapeutic or a nutraceutical. In one embodiment, the plant product may be selected from a flavonoid, alkaloid, polyphenol, polysaccharide, quinonoid and saponin.

In another aspect of at least one embodiment of the invention, there is provided a composition comprising at least one plant stem cell obtained by the method of at least one embodiment of the invention, or at least one plant stem cell extract and/or plant product obtained or obtainable from the stem cell or stem cell line of at least one embodiment of the invention. The composition may be selected from a cosmetic, agricultural, pharmaceutical, food, beverage or human/animal healthcare composition.

In a further aspect, there is provided a method of producing a plant or plant part thereof and/or a method of crop breeding, the method comprising
(a) isolating at least one stem cell derived from a tissue from a primary meristem in a leaf of a plant according to the method of claim 1;
(b) establishing a stem cell line comprising the at least one isolated stem cell, preferably wherein the stem cell line is substantially genetically homogenous;
(c) optionally selecting a high-performance cell line; and
(d) growing a new plant or plant part thereof from at least one stem cell of the stem cell line and/or at least one stem cell of the high-performance cell line.

The method may further comprise growing the plant or plant part using vertical farming practices.

A plant according to all aspects of at least one embodiment of the invention described herein may be a dicotyledon, a monocotyledon or a gymnosperm. In one embodiment the plant is a vascular plant.

### DETAILED DESCRIPTION

The present invention will now be further described. In the following passages, different aspects of embodiments of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of embodiments of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

A plant according to all aspects of at least one embodiment of the invention described herein may be a dicotyledon, a monocotyledon or a gymnosperm. In one embodiment the plant is a vascular plant.

In one embodiment, the plant is a crop plant. By crop plant is meant any plant which is grown on a commercial scale for human or animal consumption or use. In another embodiment the plant is Arabidopsis.

In one embodiment, the plant may be selected from asparagus, grasses, palms, rose, cactus, potato, tomato, coconut, broccoli, fig, sweet potato, coriander, sunflower, peanuts, strawberry, ginger, quinoa, tulip, ginger, pomegranate, aloe vera, yews (taxus) aubergine, pineapple, sumac, chickpea, rosemary, lychee, licorice (Glycyrrhiza glabra), spinach, soybean, brassicas, carrot, corn, rice, Thuja, Juniper, Pine, Garlic, cucumber, chilies, peppers, lettuce, peaches, watermelons, grapes, apples, onions, mandarins, bananas, peas, mangos, oranges, tea, sugarcane, cotton, barley, sorghum, wheat, rice, quassia and Magnolia, as well as members of the Quillija species and Cupressaceae family.

In one embodiment, the plant may be selected from a brassica, legume, cereal, citrus, root vegetable, tuber and rhizome crop, fruits including berries and soft fruits and fruit, nut and seed bearing trees.

For the purposes of this disclosure, the term “plant stem cell” refers to a plant cell capable of self-renewal and differentiating into specialized cells. Thus, through mitosis, plant stem cells are able to maintain a stem cell population and divide to generate precursor cells that subsequently differentiate into tissue and organs. This asymmetric division may occur at a cell or population level.

The terms “plant stem cells” and “stem cells” are terms used interchangeably hereafter.

By “plant stem cells” it may also be meant plant stem cells that have not previously undergone substantial differentiation, have been removed from meristematic tissue and are maintained as a substantially homogenous population under culture conditions. Thus, the stem cells described in this disclosure are distinct from plant cells that have been obtained from differentiated, somatic tissue and are stimulated or de-differentiated into a more potent state.

In one embodiment, the plant stem cells display totipotency or pluripotency. Totipotency refers to cells with the differentiation potential to give rise to all cell types and establish an entire complete organism, while pluripotency refers to cells with the differentiation potential to give rise to the majority but not all cell types.

In one embodiment, the plant stem cells are derived from primary and/or secondary meristematic tissue of plants.

By “meristematic tissue” is meant the tissue comprising populations of plant stem cells. Meristematic tissues are organized into meristems, zones of meristematic tissue that contribute to the growth of the plant. “Primary meristems” are established during embryogenesis and contribute to primary growth of a plant, namely the length or height. Examples of primary meristems include the shoot apical meristems (SAM) and the root apical meristems (RAM).

Meristems that derive from primary meristems and are established post-embryonically can be referred to as “secondary meristems”, such as axillary meristems. Secondary meristems contribute to growth in width of stems and roots in plants, resulting in thicker, sturdier tissues that can support the growing plant. Together, meristematic tissue comprises the plant stem cell niche.

In one embodiment, the plant stem cells are derived from primary meristematic tissue, preferably from primary meristematic tissue found within and/or surrounding the vasculature of a plant, more preferably the vasculature of a leaf. These stem cells are referred to herein as vascular stem cells or VSCs.

In a preferred embodiment, the plant stem cells are derived or obtained from primary meristematic tissue around or from the central vein of a leaf. The central vein of a leaf corresponds to the main vein or midrib. Accordingly, the stem cells are obtained from exposing the midrib and/or the central vein and obtaining the stem cells.

A vein in a leaf may be defined as a supporting structure throughout the leaf lamina, that provides support and structure to the leaf. Leaf veins comprise the xylem, a conductive tissue which transports water and minerals throughout the leaf and wider plant. Lead veins also comprise phloem, a tissue which transports glucose produced during photosynthesis. Accordingly, a vein may comprise xylem and/or phloem, in addition to meristematic tissue.

By other vein is meant a vein that stems from the central vein (also known as the midrib). From veins, stem venules. Accordingly, ‘other vein’ refers to a vein or venule that is not the central vein.

A phloem is formed from a protophloem that matures into a metaphloem and then finally a phloem. The cells comprising a protophloem are referred to as protophloem cells. The early phloem is formed by stem cells in the meristems, thus cells of the early protophloem may comprise meristematic stem cells, or cells sharing characteristics of both the protophloem and meristematic cells. Such cells may be referred to as protophloem meristematic cells. Protophloem meristematic cells can be defined by the presence of one or more marker cells. Examples of suitable marker cells include APL (ALTERED PHLOEM DEVELOPMENT) (Bonke et al 2003), SWEET11 (Gebauer et al 2017) and BZIP9 (basic leucine zipper 9).

In one embodiment, the leaf is selected from a plant growing or grown in a vertical farm. By a vertical farm or vertical farming is meant, cultivation practices that allow the cultivation of plant species on multiple overlapping levels, with the aim of maximizing the number of plants per cubic meter. Vertical farms are structures that produce plant species of the highest quality, since the cultivation process takes place inside a closed environment, in which all environmental parameters are controlled (temperature, humidity, CO2, light and nutrients). As such, selecting a leaf from a plant growing or grown in a vertical farm allows for highly controlled and selected cellular material for the isolation of a stem cell and/or stem cell line. Likewise, producing a plant or plant part from a stem cell using vertical farming techniques will allow for the highest quality specimens to be obtained.

### Dissecting Plant Tissue to Expose Plant Stem Cells

Prior to dissecting the plant tissue, it is subjected to a sterilization step. The sterilization step aims to remove micro-organisms from the surface of the plant to be utilized for the isolation of the target cells.

Appropriate sterilizing agents are commonly known in the technical field and may be, for example, sodium hypochlorite (bleach), calcium hypochlorite, hydrogen peroxide, ethanol, etc. Preferably, sodium hypochlorite is used for sterilization in a concentration of 0.5-1.0% and for 30-40 minutes. Calcium hypochlorite powder should be dissolved in water before use, followed by filtration. Typically, it is used in a concentration of 3.25%. Hydrogen peroxide may be used in a concentration of 3%.

Ethanol may be used for sterilization, for example, in a concentration between 70-95%.

It is essential to remove micro-organisms from the used explant, as these microbial cells grow far more rapidly than plant cells and would quickly contaminate the method of isolating cells. At the same time, useful sterilizing agents like ethanol, or bleach treatment can be toxic to plant cells at high concentrations or prolonged exposition times. Therefore, it is desirable to minimize the impact of these sterilizing agents by maintaining the shortest incubation time and, preferably, the lowest effective concentration for these chemicals throughout the sterilization procedure. Significantly, leaves have a smoother surface compared to that of stems, which facilitates a milder and more gentle sterilization procedure and increases the probability of isolating viable, healthy cell populations. Thus, when leaves are used as explants, they may be kept in the solvent for as little as 1 to 15 minutes while still obtaining the removal of most of the harmful micro-organisms and ensuring that the explant is not damaged.

Accordingly, in one embodiment, there is a method of isolating a plant stem cell from a leaf, the method comprising sterilizing at least a part of a leaf containing a central or other vein.

In another embodiment, there is a method of isolating or establishing a plant stem cell line, the method comprising sterilizing at least a part of a leaf containing a central or other vein.

Similarly, in another embodiment, there is a stem cell line obtainable by a method comprising sterilizing at least a part of a leaf containing a central or other vein.

In a preferred embodiment, the leaf is sterilized with a sterilizing agent, preferably selected from sodium hypochlorite (bleach), calcium hypochlorite, hydrogen peroxide and/or ethanol.

After being sterilized, the central or other vein of the leaf is carefully cut (dissected) to expose primary meristematic cells forming the inner vascular tissue. In a preferred embodiment, the plant stem cells are derived from primary meristematic tissue around the central vein of a leaf. The central vein of a leaf corresponds to the main vein or midrib.

In an additional embodiment, the plant stem cells may be derived from lateral or secondary veins in a leaf.

Isolating cells from the central vein has practical advantages; it is typically thicker than the lateral, secondary veins and may be easier to manipulate, allowing more cells to be exposed. Preferably, the central vein or other vein is cut along its length, thus in the case of the central vein, in a direction from the tip towards the base or from the base towards the tip of the leaf. In a preferred embodiment, the leaf is cut in two halves, along and through the central vein.

The nature of an isolated cell or cell population from meristematic tissues can be confirmed as plant stem cells by quantifying the presence or absence of morphological, metabolic and/or genetic markers within the cell population. Stem cell markers and features are well known to those in the art and described in literature (see Miyashima et al. 2013), but non-limiting examples are included herein. Morphological markers include small size, roundness, a large nucleus, scant cytoplasm and prominent nucleoli. Such features can be determined by mass cytometry, described herein. Genetic markers associated with plant stem cells include, include but are not limited to WUSCHEL-like HOMEOBOX 4 (WOX4) CLAVATA3/ESR-related 41/44 (CLE41/44), PHLOEM INTERCALATED WITH XYLEM (PXY)/TDIF RECEPTOR (TDR) and CYTOKININ RESPONSE 1 (CRE1)/WOODEN LEG (WOL)/ARABIDOPSIS HISTIDINE KINASE (AHK). The expression of stem-cell markers, and other markers of interest, can be quantified by routine methods known to those in the art. For example, reverse transcriptase-polymerase chain reaction (RT-PCR) can quantify gene expression by reverse transcribing mRNA isolated from single cells to cDNA and subsequently amplifying it by PCR. The identification of cells can then be informed by adjustments in mRNA abundance.

This careful leaf dissection process exposes meristematic cells, for example protophloem meristematic cells, in the vascular tissue derived from the primary meristem. Subsequently, these cells or an area of the explant containing the meristematic cells are collected and are subjected to a culturing step, where proliferation is induced in the presence of a suitable culture medium, such as the cell induction medium described herein.

### Culturing Isolated Cells and Establishing a Cell Line

For the purposes of this disclosure, the term “plant stem cell culture” refers to the in vitro explanting, maintenance and proliferation of plant stem cells isolated by the methods described herein. Plant stem cell cultures may refer to stem cells in the liquid and/or solid phase of culturing. Thus, plant stem cell cultures refer to stem cells within a suitable medium, such as a cell induction medium on a solid platform and/or stem cells within a liquid suspension culture. In a preferred embodiment, the plant stem cell culture comprises stem cells within a liquid suspension culture. In a preferred embodiment, plant stem cells are cultured on a solid and/or liquid media.

The culturing step according to at least one embodiment of the present invention relates to the steps carried out after the exposure of the target cells from the leaf. More specifically, a leaf of a target plant species is sterilized and the central or other vein of the leaf is dissected to expose a thin layer of desired stem cells. For example, the vein is cut to expose primary protophloem cells. These and other primary meristematic cells are self-renewing, undifferentiated and display a faster growth rate than classical differentiated cells. In the culturing step, the cut leaf is laid on a cell induction medium to promote the proliferation of the meristematic cells.

By cell induction medium is meant a media that stimulates the proliferation of cells and induces the formation of a callus. By callus is meant a visible mass of undifferentiated cells. As used herein, a callus does not comprise de-differentiated cells or is not derived from de-differentiated cells.

Culturing the exposed vein will induce the formation of a callus, a visible mass of undifferentiated cells. The length of time between explanting cells and callus formation differs according to the source of the cell. DDCs form a visible callus at and after 7 days after explanting. Vascular plant stem cells form a callus earlier than 7 days, typically at 6 days. Therefore, the stem cell populations of relevance to this disclosure produce a visible callus earlier than DDCs. Cells obtained from the central or other vein of a leaf form a visually identifiable callus within 3 to 5 days, and cells obtained from the protophloem within 6 days of culturing. Therefore, the isolated stem cells of at least one embodiment of the present invention are distinct to cells obtained from differentiated cells that have undergone dedifferentiation to form a callus.

The composition of the cell induction medium is known to the person skilled in the art and may be, for example, based on the papers by Murashige and Skoog (1962) and Gamborg, et al. (1968). Specific compositions will differ as a function of the plant species employed. The media disclosed by Murashige and Skoog is hereafter referred to as MS medium, and the media disclosed by Gamborg et al. is hereafter referred to as B5 medium.

For example, the cell induction medium comprises macro, micronutrients and/or plant hormones. In a preferred embodiment, the Cell Induction Medium contains ingredients that compose a B5 or MS medium and one or more of 2,4-D, kinetin, NAA, 6-BA and other plant growth regulators. In an even more preferred embodiment, the cell induction medium contains at least 0 to 10 mg/L 2,4-D, at least 0.01 to 1 mg/L kinetin, at least 0.01 to 1 mg/L NAA and at least 0 to 10 mg/L 6-BA.

In a further embodiment of the present invention, the specific medium composition utilized to isolate meristematic cells from the central vein or other vein of a leaf is optimized to promote the proliferation of the target cell type of the specific plant species. This is achieved by testing different concentrations and ratios of the basic components including a suitable salt base, carbon and nitrogen sources, vitamins, medium optimizing additives and plant hormones at different auxin:cytokinin ratios.

In more detail, in order to promote proliferation, the dissected explant is placed on cell inducing medium and incubated under specific temperature and light/dark conditions. Notably, the proliferation of the cells from the central or other vein is visually identifiable within 3 to 5 days of incubation, in contrast with the growth of dedifferentiated cells from other areas of the explant, which form visually identifiable callus from 7 days. In one embodiment of the present invention, the more rapidly growing protophloem cell population is identified within 6 days of incubation. Subsequently, this population of cells is excised from the explant and transferred to a new petri plate containing cell induction medium.

Next, the cells are isolated from the leaf, meaning the desired stem cells are identified and separated from the explant. That is, following callus formation, a portion of or all of the stem cells may be excised from the explant and transferred to petri dishes comprising solid and/or liquid cell induction media.

Accordingly, in one embodiment, there is provided a method of isolating a plant stem cell(s) or establishing a stem cell line, the method comprising culturing at least one stem cell from the central vein or other vein. In one embodiment, the culturing comprises culturing on solid and/or liquid cell induction media.

The stem cells may be cultured on cell induction media under specific temperature and light/dark conditions. For example, a 24 h/0 h light/dark to 0 h/24 h light/dark cycle may be employed, more preferably a 18 h/6 h light/dark cycle. At the same time, a temperature between 16-30° C. may be employed. Preferably the cells are cultured at a temperature of 25±1° C.

The above embodiments are applicable to the other methods described herein, for example a stem cell line obtainable by a method, a method for producing a plant natural product or extract etc.

Further, a suspension culture is initiated where the isolated stem cells are cultured on different petri-dishes with cell line induction medium. Optionally, high-performance cell lines are selected in response to plant immune elicitors.

Subsequently, cells may be inoculated into a glass flask containing a Suspension Initiation Medium (SIM) and are cultured in liquid to establish a plant stem cell suspension culture. Preferably, the glass flask and suspension initiation medium is sterile. Suspension initiation medium is also referred to as plant suspension medium. The flask may be incubated at 21-25° C. under appropriate light/dark conditions at an agitation rate of 110-1200 RPMs. The growth of the cultured cells may be constantly monitored using an inverted stereoscope and measured after 14 days of incubation.

Further, a suspension culture is initiated. The isolated stem cell clusters are cultured on solid cell induction media under specific temperature and light/dark conditions. For example, a 24 h/0 h light/dark to 0 h/24 h light/dark cycle may be employed, more preferably a 18 h/6 h light/dark cycle. At the same time, a temperature between 16-30° C. may be employed. Preferably the cells are kept at a temperature of about 25±1° C.

A sterile glass flask containing Suspension Initiation Medium (SIM) may be inoculated with an independent cell population. The flask may be incubated at 21-25° C. under the appropriate light/dark conditions at an agitation rate of 110-130 RPMs. The growth of the cultured cells may be constantly monitored utilizing an inverted stereoscope. The cell growth may be measured after 14 days of incubation.

The measurement of cell growth may be calculated for example by transferring 25 ml of cell suspension to a sterile 50 ml conical flask and allowing the cells to set vertically for 15 minutes. The Cell Set Volume (CSV15) is recorded and is expected to reach at least a 10-fold increase during two weeks incubation. After the growth assessment, the cells are subcultured in fresh SIM medium at a rate of 1:10 CSV15:SIM (E.g. 5 ml of CSV15 will be subcultured to a final volume of 50 ml of medium in a 250 ml sterile glass flask). The subculture process may be repeated every 14 days until reaching a 100 ml subculture volume and observing a constant growth rate in each 14-day subculture. At this stage, the established suspension culture is composed of a homogenous population of meristematic cells.

In one embodiment, cell growth is measured every 14 days of incubation.

In one embodiment, measuring cell growth comprises inoculating a known volume of suspension initiation medium (SIM) with one fifth the volume of a stem cell suspension of a known density/volume, and measuring the density occupied of the stem cells following a 14 incubation period. Thus, cell growth may be defined as an increase in cell density, for example an increase of at least 20%, 30%, 40%, 50%, 60%, 70%, 80%, 90% 100%, 150%, or 200% or more, within the 14-day culturing period.

In another embodiment, measuring cell growth comprises measuring the cell set volume at day 1 and then at day 14, wherein measuring cell set volume comprises transferring a known volume of suspension initiation medium (SIM) with at least one-fifth the volume of a stem cell suspension of a known density/volume, allowing the cells to set for 15 minutes and measuring the volume (Cell set volume, CSV) of the mixture. Thus, cell growth may be defined as an increase in CSV of the cells, for example an increase of at 50%, 100%, 150%, 200%, 250%, 300%, 350%, 400%, 450%, 500%, 600%, 700%, 800%, 900% or 1000% or more within the 14-day culturing period.

In another embodiment, measuring cell growth comprises measuring the cell number at day 1 and then at day 14. In one embodiment, cell growth is defined as an increase in total cell number, for example an increase in total cell number of at least 50%, 60%, 70%, 80%, 90% 100%, 200%, 300%, 400%, 500%, 600%, 700%, or 800% within the 14-day culturing period.

The cells contained within the established plant stem cell suspension culture flask, via the method described herein, comprise a stem cell line.

Thus, in one aspect of at least one embodiment of the invention, there is provided a culture medium that is optimized for the maintenance and/or proliferation of a population of isolated stem cells and/or stem cell line.

In one embodiment, the culture medium is optimized for maintaining a substantially homogenous population of plant stem cells in culture conditions.

In one embodiment, the culture medium is a stem cell suspension medium comprising the compositions of table 2 or table 4.

In an alternative or additional embodiment, the medium is a co-cultivation medium, comprising the plant stem cell suspension medium above supplemented with 50 μM acetosyringone.

In an additional aspect of at least one embodiment of the invention, there is provided a stem cell line established from the stem cell culture methods described above.

The high genetic stability and homogeneity of the isolated stem cell population of at least one embodiment of the invention provides a high-quality stem cell line that is absent or substantially absent unwanted genetic aberrations induced by the dedifferentiation process required by alternative methods.

Furthermore, optionally, a selection strategy may be applied, to enable the identification of cell lines that produce a high yield of target natural product. This selection strategy enables the identification of high-performance cell lines from an early stages of the cell line development process.

Accordingly, in a further aspect of at least one embodiment of the invention, there is a method for selecting a high-performance stem cell or stem cell line, the method comprising: screening for said high-performance trait in at least one single plant stem cell or cells; and selecting said single plant stem cell or cells with the high-performance trait.

In one embodiment, cells are screened and/or selected at the single-cell level.

By high-performance indicator or trait, high-performance cell or cell lines is meant a cell or cell line with an improvement in plant performance, with regard to cell size, proliferation, yield of a product or developmental potency, wherein preferably said improvement is relative to another/other cell/cells in the cell line. Therefore, the phrases ‘high-performance trait’ and ‘improvement in plant performance’ are used interchangeably.

In a preferred embodiment, the high performance trait is a high yield of target natural product in response to a plant immune elicitor. Accordingly, in one embodiment, the method further comprises treating the suspension culture with a plant elicitor and screening for the production of at least one, for example, phytochemical.

In one embodiment, the plant elicitor is a plant immune activator.

A plant immune response elicitor or activator (or just “plant elicitor”) is a molecule that can be recognized by a plant cell and triggers a plant defense response. Elicitors may be derived from a pathogen and include, as examples, oligosaccharides, peptides, glycopeptides, glycolipids, lipophilic elicitors, toxins including coronatine, and polysaccharides such as chitin and its derivatives, and the like. In a preferred embodiment, a bacterial flagellin homolog is used as an elicitor for the immune response. Immune elicitors are well-known to those in the art, and include Harpin (HrpZ), Flagellin, Cold shock proteins, Elongation factor (EF-Tu), Lipopolysaccharides (LPS), Peptidoglycan, Oligogalacturonides, Lipopeptides, Dimethylsulfide, Pseudobactin, Tri-N-alkylated benzylamine derivative (NABD), 2,4-diacetylphloroglucinol (DAPG), Oligogalacturonides, Extracellular ATP and/or DL-β-aminobutyric acid (BABA) and the like. Immune elicitors may be synthetic, for example salicylic acid and its analogues, probenazole (PBZ), 1,6-dichloro-isonicotinic acid (INA), benzothiadiazole (BTH), Tiadinil (TDL), isotianil, N-Cyanomethyl-2-chloroisonicotinamide (NCI), 3-chloro-1-methyl-1H-pyrazole-5-carboxylic acid (CMPA) and the like. Abiotic stress inducers such as for osmotic stress, may also be used to stimulate the immune response of stem cells.

In a preferred embodiment, the plant elicitor is selected from salicylic acid, methyl-jasmonate, coronatine, chitosan and/or osmotic stress inducers.

In a preferred embodiment, the phytochemical belongs to the phenolics, flavonoids and/or terpenoid family of phytochemicals.

In one embodiment, the yield of natural product in response to a plant immune response elicitor is measured. Assays to measure natural products are known to the skilled person and are described in the literature (Altemimi et al., 2017). Colorimetric assays, using reagents that undergo a measurable color change in the presence of the analyte, may be used. Well-known instruments such as High Pressure Liquid Chromatography can be used for the purification of natural products. Different varieties of spectroscopic techniques like UV-visible, Infrared, Nuclear Magnetic Resonance, and mass spectroscopy can subsequently identify the purified compounds.

In one embodiment, the abundance of a target gene transcript is measured following exposure to an elicitor. In a preferred embodiment, the transcript is selected from a phytohormone signaling associated gene, for example PATHOGEN-RELATED1 or PLANT DEFENSIN1.2. In a preferred embodiment, transcript abundance is measured using RT-qPCR. In a further embodiment, transcript abundance in response to immune elicitors is measured by RNA sequencing. In a preferred embodiment, transcript abundance is measured at the single-cell level.

In another preferred embodiment, the high performance trait is high proliferative potential. In one embodiment, screening for said high performance trait comprises measuring iodo-deoxyuridine incorporation (IdU, analogous to the fluorescent BrdU). In another embodiment, screening for said high performance trait comprises measuring cyclin B1, cyclin A, and phosphorylated histone H3 using complimentary fluorophore-antibody conjugates. The detection of such markers can be achieved through flow cytometry, or combined in mass cytometry as reported in Behbehani et al., 2012.

In another preferred embodiment, the high performance trait corresponds to cell longevity. Longevity can be screened for using a marker-assisted approach. In one embodiment, screening for said high performance trait comprises measuring the expression of telomerase and/or WOX4 and/or CLE41/44 and/or CRE1 and/or WOL and/or AHK, wherein high expression correlates to high performance.

By selecting is meant electing a stem cell or cells or stem cell line for further sub-culturing, screening and/or genetically modifying selected cells or lines.

More specifically, the present disclosure describes a method to isolate multiple suspension cultures lines and select them according to their growth rate parameters. Appropriate growth parameters and methods of measurement have been discussed above. Furthermore, the faster growing cell lines can be selected according to their natural product production capabilities. This multiple selection process results in the isolation of cell populations with rapid growth rates and high yields of the desired natural product.

In one embodiment, the selection process consists of treating the suspension culture with a plant elicitor (for example but not limited to, salicylic acid, methyl-jasmonate, coronatine and chitosan, osmotic stress inducers) and evaluating the production of specific phytochemical families, including but not limited to phenolics, flavonoids or terpenoids. Additionally, the antiradical activity (ARA) of the isolated and cultured cells is a further measure to confirm the bioactivity of the synthesized natural products.

The assays to measure these natural product families are known to the skilled person and described in the literature. However, the selection strategy in at least one embodiment of the present invention, building on the isolation and culture of the target meristematic cells, encompasses the application of colorimetric tests in tandem to evaluate the response to plant elicitors and subsequently select the cell lines that display the highest yield of the target natural product.

In another embodiment of the present invention, the concentration and composition mix of the plant elicitors can be further optimized to increase the production of the target natural product in the selected high-performance plant stem cell suspension line. Thus, after identification of the cell population with the higher production of natural products, this cell line is further subjected to secondary elicitor screening by means of testing elicitors in a range from 0.001 μM to 1,000 μM. Additionally, the growth stage of the culture at the moment of elicitation and the duration of the elicitation treatment is optimized as part of this selection process.

According to the method described in the present disclosure, cells can be cultured without going through dedifferentiation into callus. That is, the method may comprise culturing wherein the cell culture does not or does not substantially comprise dedifferentiated cells and/or protoplasts. Methods are known where cell suspension cultures are generated from different tissues and from different plant species. However, in said methods, different tissues are used as explants as a whole and do not undergo a selective dissection to isolate the veins from the leaves. The initiation of cell suspension culture from whole tissue typically is the result of, for example, epidermal, palisade, guard cells etc. undergoing a dedifferentiation process, thereby returning to pluripotent cells, which can undergo mitotic cell division. A problem with this is that the resulting dedifferentiated cells accumulate significant deleterious genetic and epigenetic modifications due to the dedifferentiation process. According to at least one embodiment of the present invention, cells surrounding the veins are isolated that have the ability to continuously divide and retain pluripotent activity without undergoing the dedifferentiation process.

In plants, only meristematic cells/stem cells found in either plant primary meristems, responsible for the growth of leaves, shoots, flowers and roots or secondary meristems, required for an increase in the diameter of stems and roots, can actively divide i.e. are mitotically active. Once meristematic cells have differentiated into specific cell types, for example, palisade, epidermal and mesophyll cells within the leaf, these differentiated cells can no longer divide. Thus, the initiation of a cell suspension culture from either whole organs or tissues is the result of the differentiated cells, present in these plant organs or tissues, undergoing a dedifferentiation process, which results in their mitotic reactivation. These dedifferentiated plant cells then regain the ability to divide. However, this dedifferentiation process results in significant deleterious genetic and epigenetic modifications, resulting in the suboptimal performance of dedifferentiated cells in suspension culture, with respect to growth, cell aggregation and production of natural products. According to at least one embodiment of the present invention, plant stem cells are isolated that innately have the ability to continuously divide and retain pluripotent activity.

In particular, at least one embodiment of the present invention is not based on the isolation of callus derived from whole leaves. The method of the present invention relies on exposing a thin layer of cells located in the periphery of the central or other vein within a leaf and the subsequent isolation of leaf stem cells which are immortal, undifferentiated, grow at a faster rate and exhibit specific morphology (higher number of amyloplasts, smaller size). The faster growth rate of these leaf stem cells allows their isolation from dedifferentiated cells, resulting in a homogenized population of cells. In addition, the method according to at least one embodiment of the present invention is fast, easy to perform and may be applied to a wide variety of plant genera.

Furthermore, the isolation of a homogenous population of stem cells allows the classification of cell lines according to their ability to produce secondary metabolites in response to an immune activator screening. This selection permits the identification of high-performance cell lines from early stages of the development process.

A main concern when isolating meristematic cells from, e.g., stems (especially woody plants) is the increased risk of contamination due to the high level of endophytes (microbes) present on the stem surface. To overcome this problem, the method according to at least one embodiment of the present invention allows the isolation of stem cell-like meristematic cells from the central vein of a leaf instead of from stems. Leaves typically have a smoother surface compared to stems, which facilitates the surface sterilization process reducing the microbial contamination rate on solid medium.

In one aspect of at least one embodiment of the invention, there is provided a plant stem cell or stem cell line obtained or obtainable by any or all of the methods described herein. Additionally, there is provided a plant, plant part or cell line obtained or obtainable by the method described herein. In one embodiment, products or extracts of the plant stem cell or stem cell line are obtained or obtainable by the methods described herein. Also provided is compositions comprising a stem cell, stem cell line, plant product and/or plant extract obtained or obtainable from the methods described herein. In one embodiment, the stem cell line has not gone through dedifferentiation into callus.

In a further aspect, there is provided a stem cell line obtained from a tissue from a primary meristem in a leaf of a plant which has the following characteristics:
a. it is formed of undifferentiated cells; and
b. the stem cells of the line are morphologically characterized by the presence of multiple amyloplasts; and
c. it is a fast-growing cell line; and
d. optionally, it has high phytochemical accumulation in response to elicitation.

By fast-growing cell line, is meant a cell line with increased growth compared to control cell, for example a differentiated leaf cell, or a dedifferentiated cell.

In an additional or alternative embodiment, the plant stem cell line comprises a substantially genetically homogenous undifferentiated cell population.

In a further aspect, there is provided a method of producing a plant or plant part and/or a method of crop breeding, the method comprising
a. isolating at least one stem cell derived from a tissue from a primary meristem in a leaf of a plant;
b. establishing a stem cell line comprising the at least one isolated stem cell, preferably wherein the stem cell line is substantially genetically homogenous;
c. optionally, selecting a high-performance cell line; and
d. growing a new plant or plant part from at least one stem cell of the stem cell line.

In one embodiment, the method further comprises growing the plant or plant part using vertical farming practices. In a preferred embodiment, the high-performance cell line is a high-yielding and/or a highly proliferating cell line.

Also provided is a composition comprising a stem cell, extract or product thereof obtained or obtainable from the above-described stem cell line.

As we have described above, the plant stem cells and cell lines presented herein display greater proliferation and genetic homogeneity relative to dedifferentiated cells and cell lines.

In a preferred embodiment, the stem cell line is obtained by the method according to at least one embodiment of the present invention.

In a further aspect of at least one embodiment of the invention, there is provided a method for producing a plant natural product or extract, the method comprising
a. isolating at least one stem cell derived from a tissue from a primary meristem in a leaf of a plant;
b. establishing a stem cell line comprising the at least one isolated stem cell, preferably wherein the stem cell line is substantially genetically homogenous;
c. optionally, selecting a high-performance cell line; and/or
d. optionally, eliciting the production or secretion of a plant natural product or extract; and
e. collecting the plant natural product or extract.

In a further aspect of at least one embodiment of the present invention, there is a composition comprising at least one plant stem cell, preferably a plurality of stem cells, and preferably from a stem cell line obtainable or obtained by the methods described herein.

In an additional or alternative embodiment, the composition may comprise at least one plant stem cell extract and/or at least one plant stem cell product, preferably from a stem cell line obtainable or obtained by the methods described herein.

In one embodiment, the composition comprises at least one plant stem cell obtained or obtainable by the methods of the disclosure, or at least one plant natural product and/or extract obtained or obtainable from the stem cell or stem cell line of at least one embodiment of the invention, and preferably by selecting a high-performance cell line. That is, the composition comprises at least one plant stem cell or cell line showing a high-performance indicator or trait, or a plant natural product and/or extract obtained from said cell or cell line.

In a preferred embodiment, the composition is for use in or as a cosmetic product. The cosmetic product may be for human or animal use. Accordingly, in one embodiment, a composition for use in a cosmetic product is disclosed, comprising at least one stem cell, preferably a plurality of stem cells and/or a plant stem cell product and/or a plant stem cell extract, preferably from a stem cell line obtainable or obtained by the methods described herein. The cosmetic composition may be formulated in any known way, e.g. as a cream, lotion, water, wash, shampoo, oils etc. The concentration of the extract is usually between 0.01%, 0.02%, 0.05%, 0.075%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 3%, 3.5%, 4%, 4.5% to 5% w/w.

It is well known in the art that many plant stem cells, extracts and products have potent anti-oxidant and/or inflammatory effects. For example, Grapes (Vitis vinifera), lilacs (Syringa vulgaris), Swiss apples (Uttwiler spatlauber) etc.

Accordingly, the composition may also comprise further components for example, phytochemicals, antioxidants and/or anti-inflammatory components. Examples of anti-oxidants include Tocopherols, Lecithin, Ascorbic acid, vitamin E, vitamin C, and coenzyme Q10, anthocyanin and curcumin and the like. Examples of anti-inflammatory compounds include aloe, turmeric, calendula extract, witch hazel, niacinamide, sea buckthorn oil, vitamins C, D and E, oatmeal or ginger. Further components may be an emulsifier and/or gel forming agent, for example glycyrrhizin. In another embodiment, the composition is for use in an agricultural composition. In one embodiment, the composition may be a mono- or multi-functional plant stem cell growth composition. Particular uses of such a composition include use in increasing water absorption, reducing excessive pesticide use and increasing crop quality and/or performance. The agricultural composition may further include, but not limited to: a plant stem cells, preferably in a liquid matrix, an antibacterial agent, starch, trace metal elements that may act as fertilizer synergist, plant growth regulators, photosynthetic agents and/or water retention agents etc. Furthermore, the plant stem cell matrix liquid may be selected from one or more of agar, polyphenols, flavonoids, nucleic acids, and protein hydrolysates. Furthermore, the antibacterial agent may be selected from one or more of amino acids, metal salts, chitosan powder, and chitin. Furthermore, the trace element fertilizer synergist may include one or more of iron, manganese, boron, zinc, copper, molybdenum, chlorine, cobalt and the like. Furthermore, the plant growth regulator includes one or more of auxin, gibberellin, chlormequat, cytokinin, and abscisic acid. Furthermore, the photosynthetic agent may be sodium bisulfite or the like. Furthermore, the water retaining agent is humic acid or the like.

In another embodiment, the composition is for use in a pharmaceutical composition. In one embodiment, the pharmaceutical composition is for use in the treatment or prevention of proliferative diseases (e.g. cancer), angiogenic and cardiovascular diseases, inflammatory diseases, osteogenic diseases, metabolic diseases, neurological diseases. Plant cell extracts for use in pharmaceutical compositions are well known within the art, and have been extensively reviewed, for example in Li, et al. Stem Cell Research & Therapy (2022) 13:472.

In another embodiment, the composition is for use in a food and/or beverage composition.

For any of the above compositions, the composition may further comprise at least one human stem cell, extract or product thereof.

Embodiments of the invention may be further described by the following non-limiting statements:

A method of isolating a stem cell line derived from a tissue from a primary meristem in a leaf of a plant, comprising the steps of:
(a) sterilizing at least a part of the leaf containing the central or other vein
(b) cutting through the central or other vein of the leaf to expose primary meristematic cells from the central or other vein inner vascular tissue; and
(c) culturing cells from said exposed cells in a cell induction medium.

By stem cell line derived from a tissue from a primary meristem in a leaf of a plant, it is understood a cell line comprising cells that are self-renewing, undifferentiated and that can produce other cells, for example having a faster growth rate and smaller size in comparison to other plant cells. In the context of the present disclosure, plant stem cells are comparable to mammalian stem cells.

The sterilizing step is a step of removing any kind of micro-organisms that would contaminate the surface of the plant explant to be utilized for the isolation of the target cells.

The sterilizing agents that can be used are common in the technical field and may be, for example, sodium hypochlorite (bleach), calcium hypochlorite, hydrogen peroxide, ethanol, etc. Preferably, sodium hypochlorite is used for sterilization in a concentration of 0.5-1.0% and for 30-40 minutes. Calcium hypochlorite powder should be dissolved in water before use, followed by filtration. Typically, it is used in a concentration of 3.25%. Hydrogen peroxide may be used in a concentration of 3%.

Ethanol may be used for sterilization, for example, in a concentration between 70-95%.

It is essential to remove as far as possible all micro-organisms from the used explant, as these microbial cells grow far more rapidly than plant cells and would quickly contaminate the method of isolating cells. At the same time, useful sterilizing agents like ethanol, or bleach treatment can be toxic to plant cells at high concentrations or prolonged exposition times. Therefore, it is desirable to minimize the impact of these sterilizing agents by maintaining the shortest incubation time and, preferably, the lowest effective concentration for these chemicals throughout the sterilization procedure. Significantly, leaves have a smoother surface compared to that of stems, which facilitates a milder and more gentle sterilization procedure and increases the probability of isolating viable, healthy cell populations. Thus, when leaves are used as explants, they may be kept in the solvent for as little as 1 to 15 minutes while still obtaining the removal of most of the harmful micro-organisms and ensuring that the explant is not damaged.

After being sterilized, the central or other vein of the leaf is carefully cut (dissected) to expose primary meristematic cells forming the inner vascular tissue. The central vein of the leaf corresponds to the main vein or midrib, having a central, linear structure, running along the length of a leaf, from the base of the leaf towards its tip. By other veins, it is understood other veins that are present in the leaf beside the central one, like the lateral or secondary veins. Preferably, the vein that is chosen for exposing the cells is the central vein. This is because typically the central vein is thicker than the lateral, secondary veins and may be easier manipulated, allowing more cells to be exposed. Preferably, the central vein or other vein is cut along its length, thus in the case of the central vein, in a direction from the tip towards the base or from the base towards the tip of the leaf. In a preferred embodiment, the leaf is cut in two halves, along and through the central vein.

This careful leaf dissection process exposes meristematic cells, for example protophloem meristematic cells, in the vascular tissue derived from the primary meristem. Subsequently, these cells are subjected to a culturing step, where proliferation is induced in the presence of an adequate cell induction medium, as explained further.

After the exposure of the target cells as explained in the previous step, the area of the explant containing the meristematic cells is subjected to a culturing step on cell induction medium.

In one embodiment of the method, the cells are not going through dedifferentiation into callus.

In a further embodiment, the culturing step comprises:
(a) isolating the primary meristematic cells from the central or other vein inner vascular tissue;
(b) initiating a suspension culture; and
(c) optionally, selecting high-performance cell lines in response to plant immune elicitors

In one embodiment of the method, the initiation of the suspension culture step comprises incubating a plate comprising the isolated primary meristematic cells in a 24 h/0 h light/dark to 0 h/24 h light/dark cycle at between 16-30° C.

In one embodiment of the method, the cell induction medium comprises macro, micronutrients and/or plant hormones.

In one embodiment of the method, the cell induction medium contains ingredients that compose a B5 or MS medium and one or more of 2,4-D, kinetin, NAA, 6-BA and other plant growth regulators.

In one embodiment of the method, the cell induction medium contains from 0 to10 mg/L 2,4-D, from 0.01 to 1 mg/L kinetin, from 0.01 to 1 mg/L NAA and from 0 to 10 mg/L 6-BA.

In a further embodiment of the present invention, the specific medium composition utilized to isolate meristematic cells from the central vein or other vein of a leaf is optimized to promote the proliferation of the target cell type of the specific plant species. This is achieved by testing different concentrations and ratios of the basic components including a suitable salt base, carbon and nitrogen sources, vitamins, medium optimizing additives and plant hormones at different auxin:cytokinin ratios.

The culturing step according to at least one embodiment of the present invention relates to the steps carried out after the exposure of the target cells. More specific, previous to culturing, a leaf of a target plant species is sterilized and the central or other vein of the leaf is dissected to expose a thin layer of desired stem cells. For example, the vein is cut to expose primary protophloem cells. These and other primary meristematic cells are self-renewing, undifferentiated and display a faster growth rate than classical differentiated cells. In the culturing step, the cut leaf is laid on a cell induction medium to promote the proliferation of the meristematic cells. Next, the cells are isolated from the leaf, meaning the desired stem cells are identified and separated from the explant. Further, a suspension culture is initiated where the isolated stem cells are cultured on different petri-dishes with cell line induction medium. Optionally, high-performance cell lines are selected in response to plant immune elicitors.

In a further aspect, the stem cell line derived from a tissue from a primary meristem in a leaf of a plant which has the following characteristics:
(a) it is formed of undifferentiated cell
(b) it is morphologically characterized by the presence of multiple amyloplasts
(c) it is a fast-growing cell line
(d) optionally, having high phytochemical accumulation in response to elicitation.

In one embodiment, the stem cell line above is obtained by any of the above methods.

A cosmetic, agricultural, pharmaceutical, food, beverage or human/animal healthcare composition comprising cells derived from a cell line according to any of the above embodiments.

### EXAMPLES

Examples according to embodiments of the invention are explained below. The isolation and induction of plant stem cells derived from a primary meristem and the subsequent production of a suspension culture, together with cell line selection is described for G. glabra, T. baccata, Q. saponaria, Q. indica but by extension, may also be utilized for any leafy plant. The following examples are offered by way of illustration, not by way of limitation.

### Example 1. Preparation of Plant Material and Isolation of Plant Stem Cells from G. glabra

Surface sterilization of plant tissue. Leaves of G. glabra plant were collected. After collecting the leaves, they were surface sterilized by 70% ethanol for 10 minutes, 10% bleach (containing 5% sodium hypochlorite) for 10 minutes and 1% bleach (containing 5% sodium hypochlorite) for 10 minutes in order. After surface sterilization, they were washed 3 to 4 times with distilled water.

Inner vascular tissue preparation from G. glabra leaf. The G. glabra leaf was placed on a hard surface horizontally. The leaf was cut vertically by scalpel or razor sharp, the cutting need to be made across the midrib or other vein of the leaf to expose the inner vascular tissue which contains the target plant stem cells. The dissected leaves were laid on the media.

After 3^{rd} to 7^{th} day of culturing the leaf explant, a visible callus was observed on the exposed main or other vein of the leaf. At this timepoint, the rest of the leaf can be removed. The proliferating meristematic stem cells from the protophloem were cultured individually on different petri-dishes with a culture medium, such as a cell line induction medium. For the purpose of cell and callus induction, MS (Murashige & Skoog medium), was used. Other usable media are known in the art, for example: B5 (Gamberg's B5 medium), WPM (Lloyed & Mccown), SM (schenk & Hildebrand medium), LP (Quoirin & Lepiovre). Application of all these media is possible. The contents of MS are described in Table 1. The cultures were grown at 25±1° C. on 16 h/8 h light/dark cycle.

TABLE 1
MS media used for culturing leaf explant that has been dissected

| | | Contents |
|---|---|---|
| | Composition | (mg/L) |
| Inorganic | NH₄NO₃ | 1650 |
| salts | H₃BO₃ | 6.2 |
| | CaCl₂ | 332.2 |
| | CoCl₂•6H₂O | 0.025 |
| | CuSO₄•5H₂O | 0.025 |
| | Na2-EDTA | 37.26 |
| | FeSO₄•7H₂O | 27.8 |
| | MgSO₄ | 180.7 |
| | MnSO₄•H₂O | 16.9 |
| | Na₂MoO₄•2H₂O | 0.25 |
| | KI | 0.83 |
| | KNO₃ | 1900 |
| | KH₂PO₄ | 170 |
| | ZnSO₄•7H₂O | 8.6 |
| Vitamin | Myo-inositol | 100 |
| | Thiamine-HCl | 10 |
| | Nicotinic acid | 1 |
| | Pyridoxine-HCl | 1 |
| Amino acid | Casein hydrosylate | 500 |
| Hormone | 6-BA | 1 |
| | Kinetin | 0.1 |
| | NAA | 0.3 |
| | 2,4-D | 3 |

| | |
|---|---|
| Sucrose | 30000 |
| Activated charcoal | 100 |
| Gelrite | 2000 |

Among the calli, white and friable calli that had good growth rates were subcultured onto new media every 14 days. The growth rate of the meristematic cells derived from the exposed main or other vein was fast, with no “browning”, and these cells could subsequently be easily selected as a stable cell line. The resulting callus from G. glabra had a white-green color due to the light conditions and was maintained as single cells or small cell clusters after six months of culture in liquid media.

Establishment of G. glabra Suspension Culture

The cells derived from exposed vein of G. glabra were cultured in a flask containing the liquid media listed in the table 2:

TABLE 2
Suspension initiation media used to
initiate and sustain stem cell line

| | | Contents |
|---|---|---|
| | Composition | (mg/L) |
| Inorganic | NH₄NO₃ | 1650 |
| salts | H₃BO₃ | 6.2 |
| | CaCl₂ | 332.2 |
| | CoCl₂•6H₂O | 0.025 |
| | CuSO₄•5H₂O | 0.025 |
| | Na2-EDTA | 37.26 |
| | FeSO₄•7H₂O | 27.8 |
| | MgSO₄ | 180.7 |
| | MnSO₄•H₂O | 16.9 |
| | Na₂MoO₄•2H₂O | 0.25 |
| | KI | 0.83 |
| | KNO₃ | 1900 |
| | KH₂PO₄ | 170 |
| | ZnSO₄•7H₂O | 8.6 |
| Vitamin | Myo-inositol | 100 |
| | Thiamine-HCl | 10 |
| | Nicotinic acid | 1 |
| | Pyridoxine-HCl | 1 |
| Amino acid | Casein hydrosylate | 500 |
| Hormone | 6-BA | 1 |
| | Kinetin | 0.1 |
| | NAA | 0.3 |
| | 2,4-D | 3 |

| | |
|---|---|
| Sucrose | 30000 |

Cells were cultured on a 120 rpm rotating shaker at 25±1° C. on 16 h/8 h light/dark cycle, with a two-week subculture interval.

### Example 2. Preparation of Plant Material and Isolation of Plant Stem Cells from Q. indica

Surface sterilization of plant tissue. Leaves of Q. indica plant were collected. After collecting the leaves, they were surface sterilized by 70% ethanol for 10 minutes, 10%
bleach (containing 5% sodium hypochlorite) for 10 minutes and 1% bleach (containing 5% sodium hypochlorite) for 10 minutes in order. After surface sterilization, they were washed 3 to 4 times with distilled water.

Inner vascular tissue preparation from Q. indica leaf. The Q. indica leaf was placed on a hard surface horizontally. The leaf was cut vertically by scalpel or razor sharp, the cutting need to be made across the midrib or other vein of the leaf to expose the inner vascular tissue which contains the target meristematic cells. The dissected leaves were laid on the media.

After the 3rd to 7th day of the culture, callus was beginning to form and expand on the exposed main vein or other vein cutting of the leaf, while the rest of the leaf can be removed.

The callus was cultured individually on different petri-dishes with cell line induction medium. For the purpose of cell and callus induction, MS (Murashige & Skoog medium), was used. Other usable media are known in the art, for example: B5 (Gamberg's B5 medium), WPM (Lloyed & Mccown), SM (schenk & Hildebrand medium), LP (Quoirin & Lepiovre). Application of all these media is possible.

The contents of MS are described in Table 1. The cultures were grown at 25±1° C. in the dark. Among the calli, white and friable calli that had good growth rate were subcultured onto new media every 14 days. The growth rate of target meristematic cells derived from the exposed main vein was fast with no browning and these cells could easily be selected as a stable cell line. The resulting callus from Q. indica has a white-yellow color was maintained as single cells or small cell clusters after six months of culture.

Establishment of Q. indica Suspension Culture

The culture derived from exposed vein of G. glabra was cultured in the flask containing the liquid media listed in Table 2.

Cells were cultured on a 120 rpm rotating shaker at 25±1° C. in the dark, with a two-week subculture interval.

### Example 3. Preparation of Plant Material and Isolation of Plant Stem Cells in T. baccata and/or Q. saponaria

Surface sterilization of plant tissue. Leaves of T. baccata and/or Q. saponaria plant were collected. After collecting the leaves, they were surface sterilized by 70% ethanol for 10 minutes, 10% bleach (containing 5% sodium hypochlorite) for 10 minutes and 1% bleach (containing 5% sodium hypochlorite) for 10 minutes in order. After surface sterilization, they were washed 3 to 4 times with distilled water.

vascular tissue preparation from T. baccata and/or Q. saponaria leaf. The T. baccata and/or Q. saponaria leaf was placed on a hard surface horizontally. The leaf was cut vertically by scalpel or razor sharp, the cutting need to be made across the midrib or other vein of the leaf to expose the inner vascular tissue which contains the plant stem cell. The dissected leaves were laid on the media.

After the 3rd to 7th day of culture, callus was beginning to form and expand on the exposed main vein cutting of the leaf, while the rest of the leaf can be removed. The callus was cultured individually on different petri-dishes with cell line induction medium.

For the purpose of cell and callus induction, B5 (Gamberg's B5 medium), was used. Other usable media are known in the art, for example: MS (Murashige & Skoog medium), WPM (Lloyed & Mccown), SM (schenk & Hildebrand medium), LP (Quoirin & Lepiovre). Application of all these media is possible.

The contents of B5 are described in Table 3. The cultures were grown at 25±1° C. in the dark.

Among the calli, white and friable calli that had good growth rate were subcultured onto new media every 14 days. The growth rate of target meristematic cells derived from the exposed main vein was fast with no browning and could easily be selected to form a stable cell line. The resulting callus from T. baccata and/or Q. saponaria was white-yellow in color and was maintained as either single cells or small cell clusters of cells after six months of culture.

TABLE 3
cell induction media (B5 media)

| | | Contents |
|---|---|---|
| | Composition | (mg/L) |
| Inorganic | (NH₄)₂SO₄ | 134 |
| salts | H₃BO₃ | 3 |
| | CaCl₂ | 113.24 |
| | CoCl₂•6H₂O | 0.025 |
| | CuSO₄•5H₂O | 0.025 |
| | Na2-EDTA | 37.3 |
| | FeSO₄•7H₂O | 27.8 |
| | MgSO₄ | 122.09 |
| | MnSO₄•H₂O | 10 |
| | Na₂MoO₄•2H₂O | 0.25 |
| | KI | 0.75 |
| | KNO₃ | 2500 |
| | NaH₂PO₄ | 130.5 |
| | ZnSO₄•7H₂O | 2 |
| Vitamin | Myo-inositol | 100 |
| | Thiamine-HCl | 10 |
| | Nicotinic acid | 1 |
| | Pyridoxine-HCl | 1 |
| Amino acid | Casein hydrosylate | 500 |
| Hormone | Kinetin | 0.5 |
| | NAA | 0.5 |
| | 2,4-D | 3 |

| | |
|---|---|
| Sucrose | 30000 |
| Activated charcoal | 100 |
| Gelrite | 2000 |

Establishment of T. baccata and/or Q. saponaria Suspension Culture

The culture derived from an exposed vein of G. glabra was cultured in a. flask containing the liquid media listed in Table 4.

TABLE 4
Suspension Initiation Media

| | | Contents |
|---|---|---|
| | Composition | (mg/L) |
| Inorganic | (NH₄)₂SO₄ | 134 |
| salts | H₃BO₃ | 3 |
| | CaCl₂ | 113.24 |
| | CoCl₂•6H₂O | 0.025 |
| | CuSO₄•5H₂O | 0.025 |
| | Na2-EDTA | 37.3 |
| | FeSO₄•7H₂O | 27.8 |
| | MgSO₄ | 122.09 |
| | MnSO₄•H₂O | 10 |
| | Na₂MoO₄•2H₂O | 0.25 |
| | KI | 0.75 |
| | KNO₃ | 2500 |
| | NaH₂PO₄ | 130.5 |
| | ZnSO₄•7H₂O | 2 |
| Vitamin | Myo-inositol | 100 |
| | Thiamine-HCl | 10 |
| | Nicotinic acid | 1 |
| | Pyridoxine-HCl | 1 |
| Amino acid | Casein hydrosylate | 500 |
| Hormone | Kinetin | 0.5 |
| | NAA | 0.5 |
| | 2,4-D | 3 |

| | |
|---|---|
| Sucrose | 30000 |

Cells were cultured on a 120 rpm rotating shaker at 25±1° C. on 16 h/8 h light/dark cycle, with a two-week subculture interval.

### Example 4. Comparison of Dedifferentiated Cells Isolated from Plant Leaves and Primary Meristematic Cells Isolated by the Method According to at Least One Embodiment of the Present Invention

A Lugol's iodine staining and microscopic analysis was performed to assess the phenotypic differences between dedifferentiated cells isolated by the production of plant cells from whole leaf organs or random pieces of leaves with the plant cells isolated and cultured from the central or other vein dissection method according to at least one embodiment of the present invention. This staining procedure enables the visualization of amyloplasts, organelles that are involved in nutrient storage, which are typically present in cells that do not undergo photosynthesis.

Samples of T. baccata and G. glabra were obtained according to the procedure depicted in the Examples 1 and 2. Logol's staining could not detect the presence of amyloplasts in either Taxus baccata or Glyzhyrryza glabra dedifferentiated cells. However, the cells obtained by the central or other vein dissection method of at least one embodiment of the present invention showed a high number of amyloplasts clearly distinguishing them from classical dedifferentiated cells.

### Example 5. Comparative Analysis of the Contamination Rate on Samples Derived from Stems and Samples Derived from Leaves as Used in at Least One Embodiment of the Present Invention

Sample explants from stems and leaves were collected from a Quassia indica tropical tree which displayed a high microbial load and high contamination rate after surface sterilization with the standard protocols. After collecting the explants, they were surface sterilized by 70% v/v aqueous solution of ethanol for 10 minutes and bleach in a concentration as shown in the table below for 10 minutes, in this order. After surface sterilization, the samples were washed 3 to 4 times with distilled water. The number of contaminated explants was obtained by counting explants with visible microbe growth. The ratio between contaminated explants and total explants is calculated in the standard way. The results are presented in the table below:

As can be seen from the table, the sterilization of the leaves leads to a very low contamination rate as compared to the treatment of the stems, in similar conditions of sterilization agents and sterilization time, making them particularly useful in a method of isolating stem cells.

Harsher sterilization conditions (higher concentration of sterilization agents, e.g. concentrated bleach solutions and/or longer sterilization times) might ameliorate the risk of contamination on stem or leaf samples, however might damage the sample and negatively affect the viability of the cells to be isolated.

TABLE 5
Comparison of contamination rate of
stem and leaves after sterilization

| | | | Contaminated | Contamination |
|---|---|---|---|---|
| Sample | Sterilization | Explants | explants | Rate |
| Stem | 70% EtOH* | 46 | 20 | 43% |
| | 10% bleach* | | | |
| | 70& EtOH | 45 | 3 | 7% |
| | 100% bleach | | | |
| Leaves | 70% EtOH | 44 | 1 | 2% |
| | 10% bleach | | | |
| | 70& EtOH | 40 | 0 | 0% |
| | 100% bleach | | | |

1. A method of obtaining at least one plant stem cell, the method comprising:
sterilizing at least a part of a leaf containing a central or other vein;
dissecting the central or other vein of the leaf to expose primary meristematic cells from the central or other vein vascular tissue; and
obtaining at least one stem cell from the central or other vein inner vascular tissue.

2. The method of claim 1, wherein the method further comprises culturing the at least one stem cell to obtain a stem cell line.

3. The method of claim 1, wherein the method does not comprise the formation of a callus obtained from or substantially comprising dedifferentiated cells.

4. The method of claim 2, wherein the cell culture does not comprise, or does not substantially comprise, dedifferentiated cells and/or protoplasts.

5. The method of claim 2, wherein the culturing further comprises:
initiating a suspension culture comprising the cultured primary meristematic cells from the central or other vein inner vascular tissue; and
optionally, selecting high-performance cell lines, preferably in response to plant immune elicitors.

6. The method of claim 5, wherein the cell culture does not comprise, or does not substantially comprise, dedifferentiated cells and/or protoplasts.

7. The method of claim 6, wherein the culturing step-further comprises:
isolating the primary meristematic cells from the central or other vein inner vascular tissue;
initiating a suspension culture comprising the cultured primary meristematic cells from the central or other vein inner vascular tissue; and
optionally, selecting high-performance cell lines, preferably in response to plant immune elicitors.

8. The method of claim 7, wherein the initiation of the suspension culture further comprises incubating a plate comprising the isolated primary meristematic cells in a 24-hour to zero hour light-dark cycle to a zero hour to 24-hour light-dark cycle at between 16-30° C.

9. A plant stem cell or plant stem cell line obtained or obtainable by the method of claim 1.

10. A plant product or stem cell extract obtained or obtainable from the plant stem cell of claim 9.

11. A plant or part thereof obtained or obtainable from the plant stem cell of claim 9.

12. A stem cell line derived from a primary meristem in a leaf of a plant, wherein the stem cell line is formed of undifferentiated cells, preferably substantially genetically homogenous cells, wherein
cells of the stem cell line have a morphology with multiple amyloplasts, wherein the stem cell line is fast-growing, and
optionally, wherein the stem cell line has a high phytochemical accumulation in response to elicitation.

13. A method for producing a plant natural product or extract, the method comprising:
isolating at least one stem cell derived from a tissue from a primary meristem in a leaf of a plant, wherein preferably the at least one stem cell is derived from primary meristematic cells from the central or other vein vascular tissue; and
culturing the at least one stem cell to establish at least one stem cell line, preferably wherein the stem cell line is substantially genetically homogenous;
optionally selecting a high-performance cell line; and/or
optionally, eliciting the production or secretion of a plant natural product or extract; and
collecting the plant natural product or extract.

14. The method of claim 13, wherein the high-performance cell line has a high-performance indicator or trait, wherein the high-performance indicator or trait selected from of the group consisting of: cell size, proliferation, yield of a product, developmental potency, and combinations thereof, and wherein preferably improvement from the high-performance indicator or trait is relative to one or more other cells in the cell line.

15. The method of claim 13, wherein the eliciting the production or secretion of a plant natural product further comprises applying at least one plant elicitor to the stem cell culture.

16. The method of claim 13, wherein the plant product or extract is selected from the group consisting of: a flavonoid, an alkaloid, a polyphenol, a polysaccharide, a quinonoid, a saponin, and combinations thereof.

17. The method of claim 1, wherein the leaf is from a plant selected from the group consisting of: a dicotyledon, a monocotyledon, a gymnosperm, or combinations thereof.

18. The method of claim 17, wherein the plant is selected from the group consisting of: asparagus, grasses, palms, rose, cactus, potato, tomato, coconut, broccoli, fig, sweet potato, coriander, sunflower, peanuts, strawberry, ginger, quinoa, tulip, ginger, pomegranate, aloe vera, yews (taxus) aubergine, pineapple, sumac, chickpea, rosemary, lychee, licorice (Glycyrrhiza glabra), spinach, soybean, brassicas, carrot, corn, rice, Thuja, Juniper, pine, garlic, cucumber, chilies, peppers, lettuce, peaches, watermelons, grapes, apples, onions, mandarins, bananas, peas, mangos, oranges, tea, sugarcane, cotton, barley, sorghum, wheat, rice, quassia and Magnolia, members of the Quillija species and Cupressaceae family, and combinations thereof.

19. A composition comprising the plant stem cell of claim 9.

20. The composition of claim 18 wherein the composition is selected from: a cosmetic composition, an agricultural composition, a pharmaceutical composition, a food, a beverage, a human healthcare composition, an animal healthcare composition, and combinations thereof.

21. A method of producing a plant or plant part thereof and/or a method of crop breeding, the method comprising:
isolating at least one stem cell derived from a tissue from a primary meristem in a leaf of a plant according to the method of claim 1;
establishing a stem cell line comprising the at least one isolated stem cell, preferably wherein the stem cell line is substantially genetically homogenous;
optionally selecting a high-performance cell line; and
growing a new plant or plant part thereof from at least one stem cell of the stem cell line and/or at least one stem cell of the high-performance cell line.

22. The plant stem cell of claim 9, wherein the leaf is from a plant selected from the group consisting of: a dicotyledon, a monocotyledon, a gymnosperm, or combinations thereof.

23. The method of claim 13, wherein the leaf is from a plant selected from the group consisting of: a dicotyledon, a monocotyledon, a gymnosperm, or combinations thereof.

24. A composition comprising the plant product or stem cell extract of claim 10.
